SEQUENCE LISTING (1 sequence)

SEQ ID NO: 1 - DNA, 696 bp, Artificial Sequence
  fluorescent protein
atggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctga

(19) United States
(12) Patent Application Publication
(10) Pub. No.: US 2021/0396759 A1
(43) Pub. Date: Dec. 23, 2021

(54) Method for Predicting Human Immune Response

(71) Applicant: OBI PHARMA, INC., Taipei City (TW)

(72) Inventors: Cheng-Der Tony Yu, Taipei City (TW); Peiwen Yu, Taipei City (TW); Ming-Chen Yang, Taipei City (TW); Yu-Ching Lin, Taipei City (TW); Chien-Chih Ou, Taipei City (TW)

(73) Assignee: OBI PHARMA, INC., Taipei City (TW)

(21) Appl. No.: 17/053,934

(22) PCT Filed: May 13, 2019
(86) PCT No.: PCT/US2019/031960

§ 371 Date: Nov. 9, 2020

Related U.S. Application Data
(60) Provisional application No. 62/670,105, filed on May 11, 2018.

(51) Int. Cl. G01N 33/574; G01N 33/50

(52) U.S. Cl. CPC G01N 33/57492; G01N 33/5011

(57) ABSTRACT

The present invention relates to a method for predicting human immune response to therapeutic cancer vaccine. This method includes a series of culturing procedures and a modified ELISPOT assay to detect total antibody, antigen specific antibody, and cytokine induction ability from human individual's PBMC.

### RELATED APPLICATIONS

The present application claims priority of United States provisional application having Ser. No. 62/670,105 filed May 11, 2018, which is hereby incorporated by reference herein for all purposes.

### TECHNICAL FIELD

The present invention relates to a method for predicting human immune response to therapeutic cancer vaccine. This method includes a series of culturing procedures and a modified ELISPOT (Enzyme-Linked ImmunoSpot) assay to detect antigen specific antibody producing ability from human individual's PBMC.

### BACKGROUND OF INVENTION

Therapeutic cancer vaccine is an emerging concept that can provide long term protection by stimulating patient's own immune response. Like any other treatments that rely on patient's immune response, such as PD-1/PD-L1, it is crucial to identify responsive population that will benefit from the treatment. In regards to cancer vaccine, whether the patient is able to generate antigen specific antibody would be an important indicator.

Numerous surface carbohydrates are expressed in malignant tumor cells. For example, the carbohydrate antigen Globo H (Fucα1→2 Galβ1→3 GalNAcβ1→3 Galα1→4 Galβ1→4 Glc) was first isolated as a ceramide-linked Glycolipid and identified in 1984 from breast cancer MCF-7 cells. (Bremer E G, et al. (1984) J Biol Chem 259:14773-14777). Previous studies have also shown that Globo H and stage-specific embryonic antigen 3 (Galβ1→3GalNAcβ1→3Galα1→4Galβ1→4Glcβ1) (SSEA-3, also called Gb5) were observed on breast cancer cells and breast cancer stem cells (WW Chang et al. (2008) Proc Natl Acad Sci USA, 105(33): 11667-11672). In addition, SSEA-4 (stage-specific embryonic antigen-4) (Neu5Acα2→3Galβ1→3GalNAcβ1→3Galα1→4Galβ1→4Glcβ1) has been commonly used as a cell surface marker for pluripotent human embryonic stem cells and has been used to isolate mesenchymal stem cells and enrich neural progenitor cells (Kannagi R et al. (1983) EMBO J, 2:2355-2361). These findings support that Globo series antigens (Globo H, SSEA-3 and SSEA-4) are unique targets for cancer therapies and can be used to direct therapeutic agents to targeting cancer cells effectively.

### SUMMARY

One of the objective of the present disclosure is to provide a method to evaluate responsivity of a subject to a carbohydrate antigen or its immunogenic fragment before actually administering the carbohydrate antigen to the subject. Another objective is to determine the therapeutic efficacy of a cancer vaccine to a subject.

In order to achieve the objective, the present invention provides a method of obtaining information that can be used to determine and predict the humoral immune response of patient suspected of having cancer for a carbohydrate antigen, comprising the steps of: (a) obtaining a sample from the patient, (b) cultivating a cell from the sample, (c) exposing the cell ex vivo to one or more antigens, (d) identifying the patient is a responder or a non-responder; and (e) continuing a further treatment if the patient is a responder.

The present disclosure provides a method for evaluating responsivity of a subject to a carbohydrate antigen or its immunogenic fragment, comprising a. obtaining a peripheral blood mononuclear cell from the subject; b. exposing the cell ex-vivo with the carbohydrate antigen or its immunogenic fragment in an Enzyme-Linked ImmunoSpot assay performed by using carbohydrate antigen-coated plate; and c. determining a biological activity according to the assay to evaluate the responsivity.

The method of the present disclosure is an ex-vivo immunogenicity method on human peripheral blood mononuclear cells (PBMC) to monitor antigen specific antibody production induced by glycan targeting cancer vaccine. The method described here comprises exposure procedures to stimulate PBMC with carbohydrate antigen, following by ELISPOT assay to evaluate responsivity. The spot number can represent the immune response of a subject, which can be used to evaluate the responsivity of the subject to the carbohydrate antigen.

The present disclosure relates to Globo series antigens (Globo H, SSEA-3 and SSEA-4) vaccine, including pharmaceutical compositions comprising adjuvant.

The present disclosure also provides a method for determining the therapeutic efficacy of a cancer vaccine, comprising: (a) providing a sample from a subject; (b) cultivating a cell collected from the sample; (c) contacting the cell and assaying the binding of antibodies bound to the antigens; and (d) determining the therapeutic effect of the antineoplastic agent.

### BRIEF DESCRIPTION OF THE FIGURES

A more complete understanding of the invention may be obtained by reference to the accompanying drawings, when considered in conjunction with the subsequent detailed description. The embodiments illustrated in the drawings are intended only to exemplify the invention and should not be construed as limiting the invention to the illustrated embodiments.

FIG. 1. The scheme of the ex vivo immunogenicity assay procedures, illustrating cancer vaccine treatment and culturing procedure to un-immunized human peripheral blood mononuclear cells.

FIG. 2. The comparison of conventional and modified anti-Globo H IgM detecting ELISPOT (Group A: No antigen; Group B: B-Poly-S™; Group C: OBI-821 adjuvant; Group D: OBI-821 adjuvant+OBI-822 vaccine; Group E: medium only). Each treatment are seeded in triplicate.

FIG. 3. The method accuracy of modified anti-Globo H IgM ELISPOT. FIG. 3A illustrated the transfection efficiency (PMBC only, Zsgreen only and co-transfect) by flow cytometry analysis on the seeding day (Day 11). FIG. 3B illustrated anti-Globo H IgM ELISPOT performed with different seeding CHO cell transfections. The seeding CHO cell numbers were ranging from 0 to 5×10⁴ cell/per well. Each treatment are seeded in triplicate. FIG. 3C illustrated the quantitative result of anti-Globo H IgM ELISPOT.

FIG. 4. Total IgM, anti-Globo H IgM, and IFN-γ fold change after applying OBI-833/821 immunization procedures to patient PBMC. FIGS. 4A, B, and C illustrated the fold change of total IgM ELISPOT, anti-Globo H ELISPOT, and IFN-γ induction compared to no-antigen treatment group, respectively.

FIG. 5. The correlation between ex vivo immunogenicity assay results and patient clinical anti-Globo H IgM response. FIGS. 5A, B, and C illustrated the correlation between total IgM ELISPOT, anti-Globo H IgM ELISPOT, or IFN-γ induction and patient maximum anti-Globo H IgM response after OBI-833/821 immunization, respectively.

FIG. 6. The correlation between ex vivo immunogenicity assay results and patient clinical anti-Globo H IgG response. FIGS. 6A, B, and C illustrated the correlation between total IgM ELISPOT, anti-Globo H IgM ELISPOT, or IFN-γ induction and patient maximum anti-Globo H IgG response after OBI-833/821 immunization, respectively.

### DETAILED DESCRIPTION OF THE INVENTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

The present invention provides a method of obtaining information that can be used to determine and predict the humoral immune response of patient suspected of having cancer for a carbohydrate antigen, comprising the steps of: (a) obtaining a sample from the patient, (b) cultivating a cell from the sample, (c) exposing the cell ex vivo to one or more antigens, (d) identifying the patient is a responder or a non-responder; and (e) continuing a further treatment if the patient is a responder.

The present disclosure discloses a method for evaluating responsivity of a subject to a carbohydrate antigen or its immunogenic fragment. The method comprises a. obtaining a peripheral blood mononuclear cell from the subject; b. exposing the cell ex-vivo with the carbohydrate antigen or its immunogenic fragment in an Enzyme-Linked ImmunoSpot assay performed by using carbohydrate antigen-coated plate; and c. determining a biological activity according to the assay to evaluate the responsivity.

The present disclosure also provides a method for determining the therapeutic efficacy of a cancer vaccine, comprising: (a) providing a sample from a subject; (b) cultivating a cell collected from the sample; (c) contacting the cell and assaying the binding of antibodies bound to the antigens; and (d) determining the therapeutic effect of the antineoplastic agent.

The term “patient” used herein is not limited to a person who has the disease (ex. cancer) for sure but also includes those who are suspected of having the disease. In a preferable embodiment, the further treatment could involve the usage of the antigen (carbohydrate antigen).

The term “responder” used herein is referred to a person who is in response to the exposure of the carbohydrate antigen to an extend that exceeds a threshold value. The threshold value (which in some circumstance can be considered as a cut-off value) could vary depending on the type of disease, the average biological activity of a peer group, the medical practitioner's opinion, etc. The tem of “non-responder” is defined on the contrary of “responder” as above.

The term “responsivity” is referred to the degree of the subject in response to the exposure of the carbohydrate antigen. In an embodiment, the responsivity represents a biological activity induced directly or indirectly by the carbohydrate antigen. The term “biological activity” can be defined by a biological change in the product of any effector molecules, including but not limited to IgM, IgM against the carbohydrate antigen, cytokine, or a combination thereof. In an embodiment, the responsivity can have therapeutic meaning. In a specific embodiment, the responsivity can be applied to determine the therapeutic efficacy of the carbohydrate antigen to the subject. In a preferable embodiment, the responsivity is evaluated by comparing the biological activity with a threshold value and the subject is identified as responsive to the carbohydrate antigen if the biological activity exceeds the threshold value. In a specific embodiment, the biological activity is determined by spot number of the Enzyme-Linked ImmunoSpot assay.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

In an embodiment, the carbohydrate antigen-coated plate is a plate without PVDF. In a specific embodiment that the carbohydrate antigen is a Globo series antigen, the carbohydrate antigen-coated plate is a Globo series antigen-lipid coated plate. In an exemplary embodiment, the carbohydrate antigen-coated plate is commercially available flat white plate.

In a preferable embodiment, the carbohydrate antigen or its immunogenic fragment is a Globo series antigen. Numerous surface carbohydrates are expressed in malignant tumor cells. For example, the carbohydrate antigen Globo H (Fucα1→2 Galβ1→3 GalNAcβ1→3 Galα1→4 Galβ1→4 Glc) was first isolated as a ceramide-linked Glycolipid and identified in 1984 from breast cancer MCF-7 cells. (Bremer E G, et al. (1984) J Biol Chem 259:14773-14777). Previous studies have also shown that Globo H and stage-specific embryonic antigen 3 (Galβ1→3GalNAcβ1→3Galα1→4Galβ1→4Glcβ1) (SSEA-3, also called Gb5) were observed on breast cancer cells and breast cancer stem cells (WW Chang et al. (2008) Proc Natl Acad Sci USA, 105(33): 11667-11672). In addition, SSEA-4 (stage-specific embryonic antigen-4) (Neu5Acα2→3Galβ1→3GalNAcβ1→3Galα1→4Galβ1→4Glcβ1) has been commonly used as a cell surface marker for pluripotent human embryonic stem cells and has been used to isolate mesenchymal stem cells and enrich neural progenitor cells (Kannagi R et al. (1983) EMBO J, 2:2355-2361). These findings support that Globo series antigens (Globo H, SSEA-3 and SSEA-4) are unique targets for cancer therapies and can be used to direct therapeutic agents to targeting cancer cells effectively.

In a preferable embodiment, DT is diphtheria toxin cross-reacting materials (DT-CRM) or diphtheria toxoids. A DT-CRM refers to a mutant diphtheria toxin, e.g., by mutation or by chemical modification, such that it no longer possesses sufficient ADP-ribosyl. Non limiting examples of DT-CRM include DT-CRM 30, DT-CRM 45, DT-CRM 176, DT-CRM 197 and DT-CRM 228. A diphtheria toxoid is a formaldehyde-inactivated diphtheria toxin. DT is commercially available from or can be prepared by methods known in the art, such as recombinant DNA technology as described in U.S. Pat. No. 5,614,382, the content of which is incorporated by reference in its entirety.

In a specific embodiment, the carbohydrate antigen or its immunogenic fragment is conjugated with a carrier protein; wherein, the carrier protein can be, but not limited to KLH (Keyhole limpet hemocyanin), DT-CRM 197 (diphtheria toxin cross-reacting material 197), or a combination thereof.

Practically, the carbohydrate antigen might be formulated with an adjuvant into a cancer vaccine. The adjuvant comprises, but not limited to, saponin, Freund's adjuvant, a-galactosyl-ceramide (α-GalCer) adjuvant, aluminum phosphate, aluminum hydroxide, MF59 (4.3% w/v squalene, 0.5% w/v polysorbate 80 (Tween 80), 0.5%>w/v sorbitan trioleate (Span 85)), CpG-containing nucleic acid, QS21 (saponin adjuvant), a -Galactosyl-ceramides or synthetic analogs thereof (e.g., C34, see U.S. Pat. No. 8,268,969), MPL (Monophosphoryl Lipid A), 3DMPL (3-O-deacylated MPL), extracts from Aquilla, ISCOMS (see, e.g., Sjolander et al. (1998) and J. Leukocyte Biol. 64:713), LT/CT mutants, poly(D,L-lactide-co-glycolide) (PLG) microparticles, Quil A, QS-21 (a saponin from Quillaja saponaria tree bark), interleukins, Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(-2′-dip-almitoyl-sn-glycero-3-hydroxyphosphoryloxy)-ethylamine (CGP 19835 A, referred to as MTP-PE), and RIBI, which contains three components extracted from bacteria, monophosphoryl lipid A, trehalose dimycolate and cell wall skeleton (MPL+TDM+CWS) in a 2%>squalene/Tween 80 emulsion, or a combination thereof.

In a specific embodiment, the cancer vaccine is applied for preventing or treating Globo series antigen expressing cancer including, but are not limited to, sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testical cancer, buccal cancer, oropharyngeal cancer, laryngeal cancer and prostate cancer.

Specifically, the cancer vaccine can be OBI-822/821, OBI-833/821, OBI-833/834, OBI-866/821 or OBI-866/834. Exemplary OBI-822 (Globo H-KLH glycoconjugate) is as described in WO2015/159118, WO2016/044326 and WO2017/185089 patent applications, the contents of which are incorporated by reference in its entirety. Exemplary OBI-833 (Globo H-DT glycoconjugate) is as described in WO2010/005598 and WO2014/107652 patent applications, the content of which is incorporated by reference in its entirety. Exemplary OBI-866 (SSEA4-KLH glycoconjugate) is as described in WO2018/022933 patent application, the content of which is incorporated by reference in its entirety. Exemplary OBI-821 is a saponin adjuvant and OBI-834 is a α-galactosyl-ceramide (α-GalCer) adjuvant.

In certain embodiment, the therapeutic cancer vaccine is OBI-822 (Globo H-KLH glycoconjugate, OBI Pharma, Inc.) combined with adjuvant OBI-821 (saponin adjuvant, OBI Pharma, Inc.) were treated to the human PBMC for their immunogenicity ability test, especially the ability of antigen specific anti-Globo H IgM and IgG induction.

In certain embodiment, the therapeutic cancer vaccine is OBI-833 (Globo H-DT glycoconjugate, OBI Pharma, Inc.) combined with adjuvant OBI-821 (saponin adjuvant, OBI Pharma, Inc.) were used to treat the human PBMC to observe their ability to stimulate the production of immunoglobulins, especially the ability of antigen specific anti-Globo H IgM and IgG induction.

In certain embodiment, the sample is a body fluid (serum, saliva, lymph node fluid, urine, vaginal swab, or buccal swab) collected from patient. In an embodiment, the term “vaccine”, “cancer vaccine” and “antineoplastic agent” are interchangeable in this specification.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: The Scheme of Experimental Procedure

Culture of Human PBMC

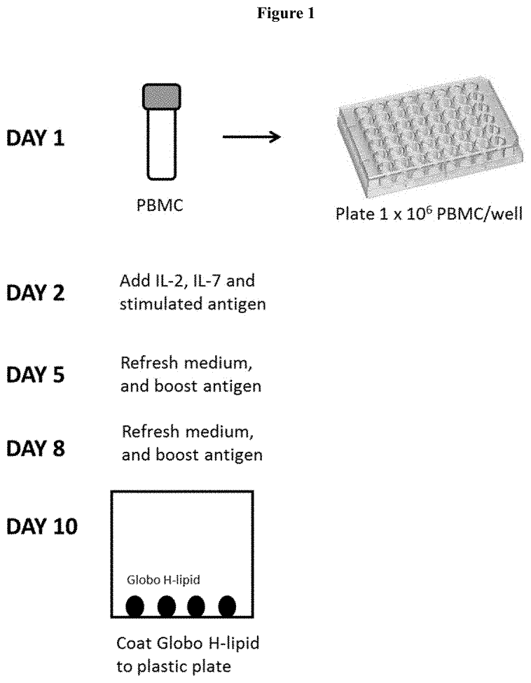

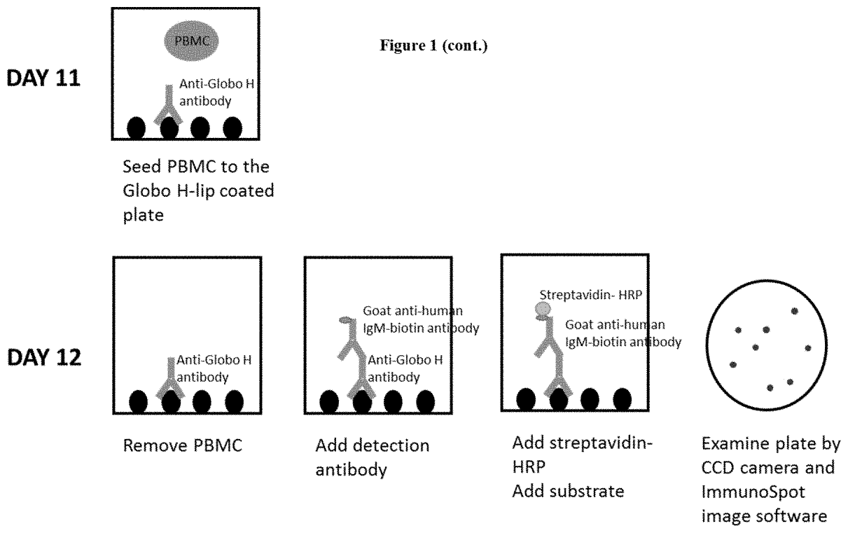

The overall treatment procedure scheme is shown in FIG. 1. Frozen vial of human PBMC, either from healthy donor or harvested form patients, was thawed and immediately washed once by complete culture medium (RPMI-1640 with 10% FBS, 1% sodium pyruvate, 1% L-glutamine, 0.1% 2-ME, 1% non-essential amino acid, and 1% penicillin-streptomycin). Cell pallet was resuspended in complete culture medium and counted for cell number and cell viability. Upon confirmation of cell viability greater than 80%, seeding of PBMC into 48-well culture plate (Catalog#150687; Thermo Fisher Scientific Inc.) in the cell density of 1×10⁶ cell/per well containing 0.5 mL of complete culture medium was performed. Culture plate was incubated at 37° C., 5% CO₂ overnight. On Day 2, equal volume of antigen and IL-2, IL-7 containing medium were added into each well. Incubate the plates at 37° C. until use. On Day 5, refresh of the medium with antigen and cytokines was performed, then incubate the plates at 37° C. until use. On Day 8, seeding the cells and PBMC was collected and transferred into new 48-well plate at 37° C., 5% CO₂.

Measurement of Total IgM

Total IgM induction status was evaluated by commercially available total IgM ELISPOT kit (Cellular Technology Ltd.). On Day 10, a 96-well plate coated with human Ig capture antibody was incubated at 4° C. overnight. Plate was washed once with PBS and used for seeding of the treated PBMC at 2.5×10⁴ cells per well density. Incubate the plates at 37° C. overnight, avoid shaking. On Day 12, plate was washed twice with PBS, followed by two wash cycles with 0.05% PBST (0.05% Tween-20 in PBS). 80 μL of biotin-labeled anti-IgM detection antibody were added into each well and incubated at room temperature for 2 hours. Plate was washed three times with 0.05% PBST and 80 μL of Tertiary solution were added into each well. Incubation at room temperature was conducted for 1 hour. Plate was washed twice with 0.05% PBST followed by two wash cycles with ddH₂O. Plate was added with 80 μL of mixed blue developer solution per well, and incubated at room temperature for 15 minutes while avoiding light exposure. Plate was rinsed with tap water and put in inverted position to air-dry. Imaging of the 96-well plate was performed using a CCD video camera, and the spot number was determined by ImmunoSpot® software (Cellular Technology Ltd.).

Measurement of Anti-Globo H IgM

Anti-Globo H IgM was evaluated by commercially available total IgM ELISPOT kit (Cellular Technology Ltd.). On Day 10, a flat bottom 96-well plate (Catalog #3917; Corning Incorporated) was coated with 0.2 μg Globo H-lipid (C₇₈H₁₄₄N₂O₃₃, MW 1637.9716) in 100 μL ethanol per well. Plate was incubated at room temperature overnight where the ethanol was completely evaporated. Plate was added with 200 μL of complete culture medium per well and incubated at room temperature for 1 hour.

Plate was used for seeding of the treated PBMC at 2×10⁵ cells per well density. Incubate the plates at 37° C. overnight, avoid shaking. On Day 12, plate was washed twice with PBS, followed by two wash cycles with 0.05% PBST. Each well of the plate was added with 100 μL of filtered goat anti-human IgM-biotin antibody (Catalog#109-067-043; Jackson ImmunoResearch Inc.) solution in 1% BSA dissolved in PBS at 1:1000 dilution. Plate was incubated at room temperature for 2 hours. Plate was washed with 0.05% PBST three times. Each well was added with 100 μL of streptavidin-HRP (Catalog#DY998; R&D Systems, Inc.) in 1% BSA buffer at 1:200 dilution. Plate was incubated at room temperature for 1 hour. Two wash steps with 0.05% PBST followed by two washes of ddH₂O were performed. Plate was added with 100 μL of mixed substrate solution (Catalog#CTL-STR10; Cellular Technology Ltd.) per well, and incubated at room temperature for 15 minutes while avoiding light exposure. Plate was rinsed with tap water and put in inverted position to air-dry. Imaging of the 96-well plate was performed using a CCD video camera, and the spot number was determined by ImmunoSpot® software (Cellular Technology Ltd.).

Measurement of IFN-γ

On Day 11, PBMC cultured supernatant were collected into a 15 mL tube (Catalog #430791; Corning Incorporated), and centrifuged at 400 g at room temperature for 5 minutes. Supernatant was transferred into a new 1.5 mL microtube (Cataloge #MCT-150-C; Corning Incorporated), and stored at −80° C. IFN-γ was measured by MSD V-PLEX human proinflammatory cytokine kit (Catalog #K151A0H-2; Meso Scale Diagnostic, LLC). The experimental procedures were performed according to manufacturer's instructions. The results were acquired by MESO SECTOR S600 (Meso Scale Diagnostic, LLC).

### Example 2: The Modified Anti-Globo H IgM ELISPOT Assay

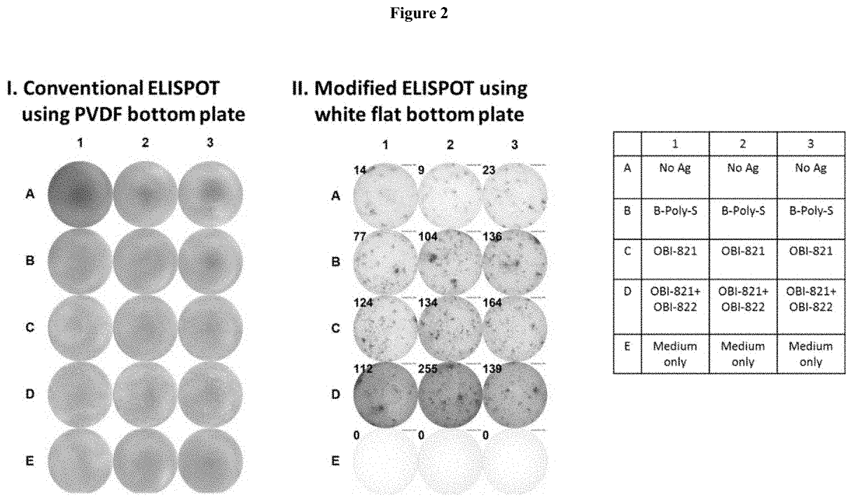

The conventional and modified ELISPOT assay were compared in parallel (Group A: No antigen; Group B: B-Poly-S™ (Catalog #CTL-hBPOLYS-200; Cellular Technology Ltd.); Group C: OBI-821 adjuvant; Group D: OBI-821 adjuvant+OBI-822 vaccine; Group E: medium only). Conventional and modified ELISPOT assays were performed using PVDF bottom 96-well plate (Catalog #MSIPS4510; Merck KGaA) and flat white bottom 96-well plate, respectively. The condition of coating and seeding of PMBC cell number for both assays were identical. FIG. 2 illustrates higher visibility and sensitivity of modified ELISPOT assay when compared to conventional method.

### Example 3: The Accuracy of Modified Anti-Globo H IgM ELISPOT

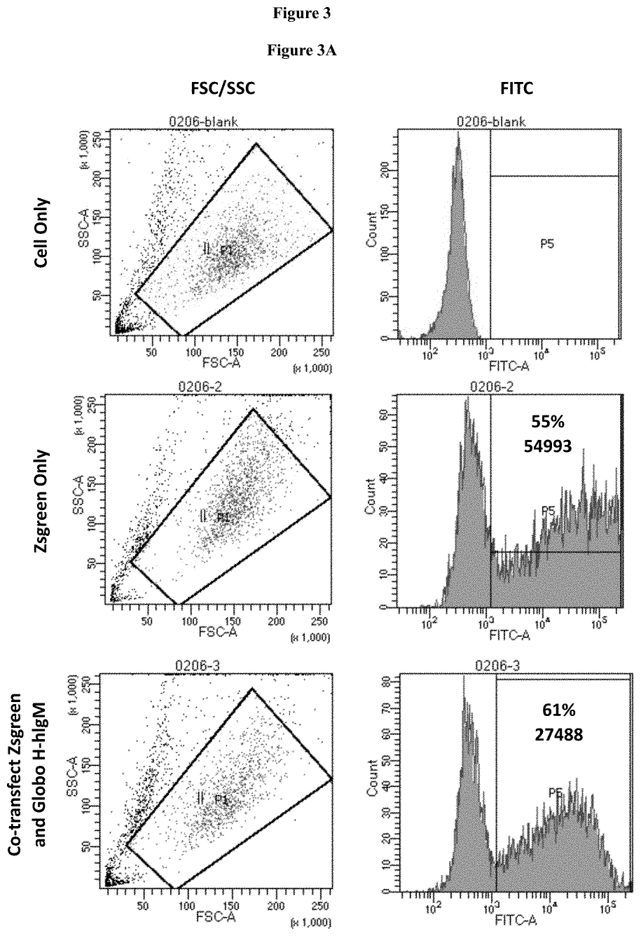

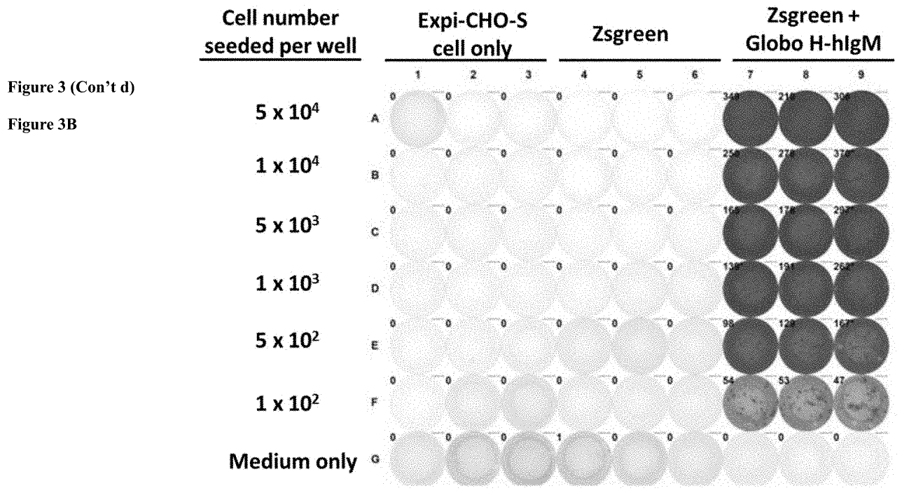

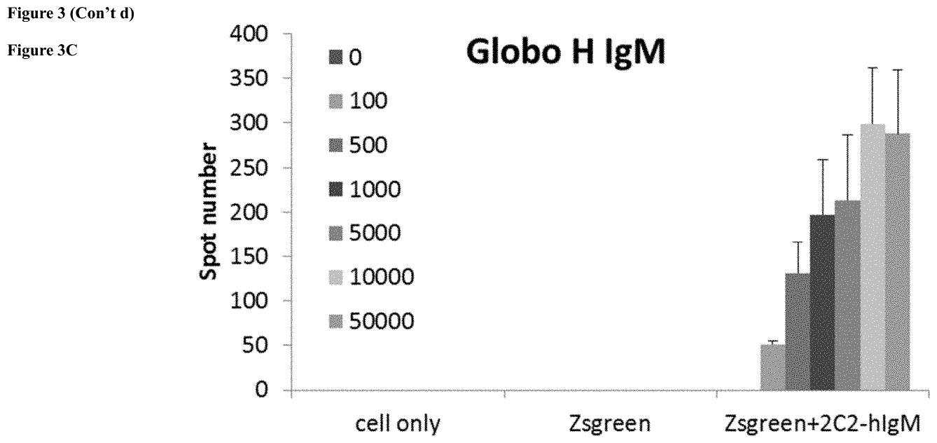

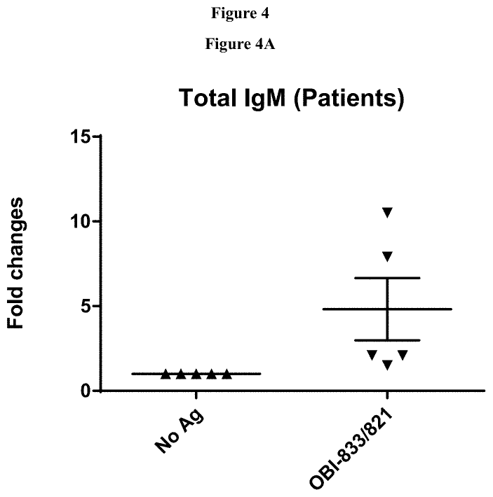

The ExpiCHO-S cells (Catalog #A29127; Thermo Fisher Scientific Inc.) were cultured in ExpiCHO expression medium. When cells density reached 4×10⁶ to 6×10⁶ viable cells/mL, 1.8×10⁸ cells were co-transfected by ExpiFectamine CHO transfect kit with 15 μg of plasmid Zsgreen and anti-human GloboH-IgM antibody in 30 mL ExpiCHO expression medium. After 8 days of transfection, cells were collected for assay. Three types of cells, including CHO cell only, CHO cells transfected with Zsgreen fluorescent protein sequence (SEQ ID No. 1), and CHO cells co-transfected with Zsgreen and human Globo H IgM, were seeded for anti-Globo H IgM ELISPOT assay. FIG. 3A illustrates the transfection efficiency of 55 to 61% on the seeding day (Day 11) by Flow Cytometry. FIGS. 3B and 3C illustrate that only the CHO cells co-transfected with Zsgreen and human Globo H IgM had detectible spots, which confirm the selectivity and accuracy of the modified Globo H IgM ELISPOT assay.

### Example 4: Serum Anti-Globo H IgM or IgG Concentration of OBI-833/821 Vaccinated Patients

Patients were immunized with OBI-833/821 in a phase 1 clinical study (Trial Identifier #NCT02310464). Anti-Globo H IgM and IgG concentration were measured by glycan chip. Maximum serum Globo H IgM and IgG concentration (μg/mL) of each sample are summarized in Table 1. Patients' anti-Globo H IgM and IgG concentration in serum will be used to compare with ex vivo immunogenicity assay results.

TABLE 1
Serum anti-Globo H IgM or IgG concentration of
OBI-833/821 vaccinated patients.

OBI-833/821

| | Clinical Globo H IgM | Clinical Globo H IgG |
|---|---|---|
| Patient ID | (μg/mL) | (μg/mL) |
| 001-004 | 6.19 | 9.11 |
| 034-002 | 57.21 | 5.06 |
| 034-004 | 242.81 | 4.15 |
| 034-006 | 10.63 | 13.72 |
| 034-007 | 25.36 | <LLOQ * |

* LLOQ: Lower Limit of Quantification.

### Example 5: Ex Vivo Immunogenicity Assay Result from Patient PBMC

Total IgM and Globo H IgM Response

Patient PBMC was collected and cryopreserved prior to OBI-833/821 vaccination. Ex vivo immunogenicity assay was performed following protocols illustrated in FIG. 1. PBMC from known healthy donor was used as experimental control. Spot number and fold change value normalized with no-antigen treatment group of total IgM and anti-Globo H IgM by ELISPOT assay are summarized in Table 2.

TABLE 2
Total IgM and anti-Globo H IgM ELISPOT
result from patient PBMC.

| | Spot Number | | | | Fold changes | | | |
|---|---|---|---|---|---|---|---|---|
| | Total IgM | | Globo H IgM | | Total IgM | | Globo H IgM | |
| | ELISpot | | ELISpot | | ELISpot | | ELISpot | |
| | | OBI- | | OBI- | | OBI- | | OBI- |
| Patient | No | 833 + | No | 833 + | No | 833 + | No | 833 + |
| ID | Ag | 821 | Ag | 821 | Ag | 821 | Ag | 821 |
| 001-004 | 21 | 31.5 | 8 | 78 | 1 | 1.50 | 1 | 9.75 |
| 034-002 | 5.3 | 42 | 38 | 65 | 1 | 7.92 | 1 | 1.71 |
| 034-004 | 17 | 179 | 9.6 | 123 | 1 | 10.53 | 1 | 12.81 |
| 034-006 | 53 | 111.6 | 14.3 | 30 | 1 | 2.11 | 1 | 2.10 |
| 034-007 | 22.5 | 46.6 | 10.5 | 18.5 | 1 | 2.07 | 1 | 1.76 |

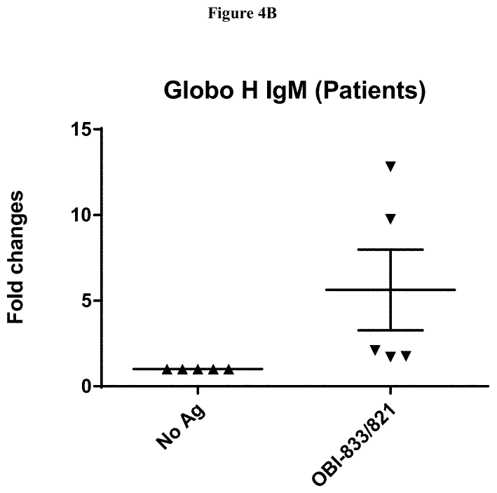

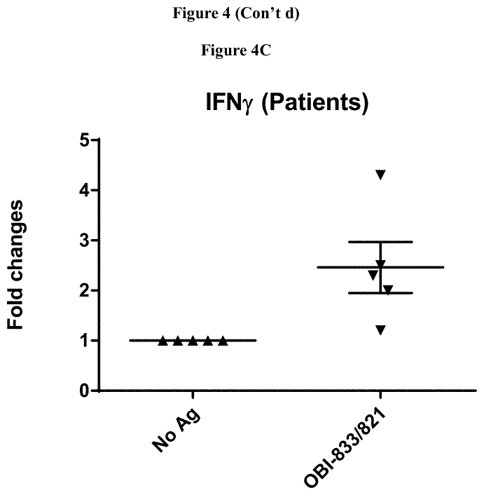

Total IgM and Globo H IgM responses from five patients' PBMC are illustrated in FIGS. 4A and 4B, respectively. The results indicated that with ex vivo immunogenicity culturing procedures, various levels of total IgM and/or Globo H IgM were induced using patients' PBMC.

IFN-γ Response

In addition to antibody responses, cytokine production, such as IFN-γ, was evaluated using ex vivo immunogenicity culturing procedures. IFN-γ concentration (pg/mL) detected in the culture supernatant and fold change normalized with no-antigen treatment group are illustrate in Table 3 and FIG. 4C, respectively. The results indicated that with ex vivo immunogenicity culturing procedures, various levels of IFN-γ were induced using patients' PBMC

TABLE 3
IFN-γ induction result from patient PBMC.

| | IFN-γ (pg/mL) | | Fold changes | |
|---|---|---|---|---|
| Patient ID | No Ag | OBI-833 + 821 | No Ag | OBI-833 + 821 |
| 001-004 | 18795 | 23142 | 1 | 1.23 |
| 034-002 | 10054 | 24715 | 1 | 2.46 |
| 034-004 | 22915 | 99184 | 1 | 4.33 |
| 034-006 | 15754 | 35523 | 1 | 2.25 |
| 034-007 | 4396 | 8680 | 1 | 1.97 |

### Example 6: Correlation Between Ex Vivo Immunogenicity Assay Results and Clinical Response

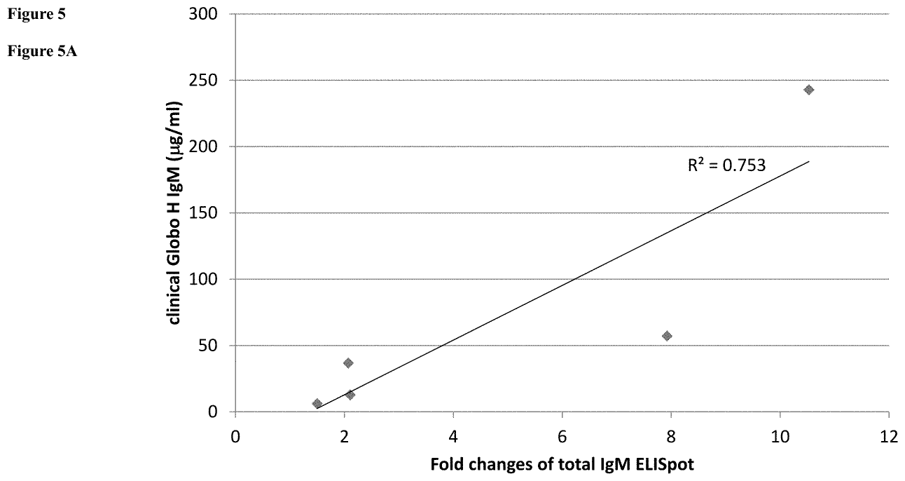

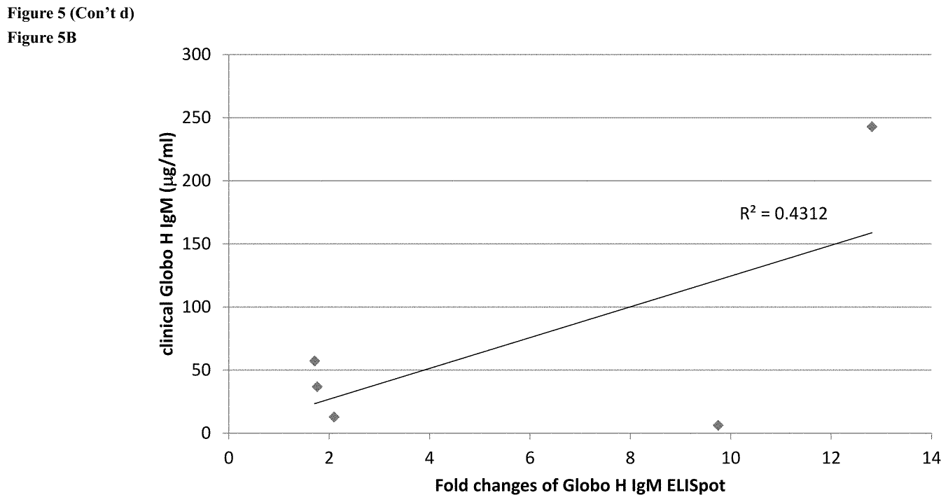

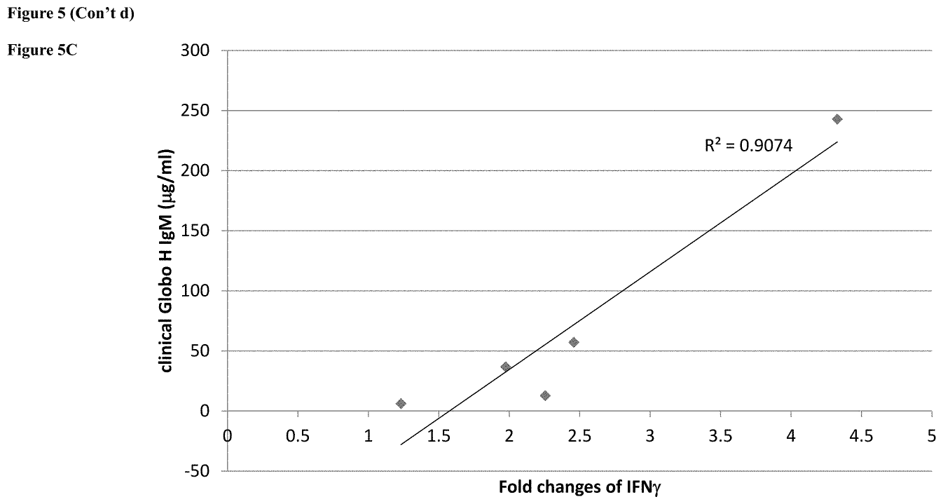

The correlation of patients' maximum serum anti-Globo H IgM concentration (μg/mL) in comparison with either total IgM, anti-Globo H IgM, and IFN-γ cytokine induction are presented in FIGS. 5A, 5B, and 5C, respectively. ELISPOT result as well as IFN-γ induction are shown in fold change normalized with no-antigen treatment group. Positive correlation between results and patients' serum anti-Globo H IgM level were observed.

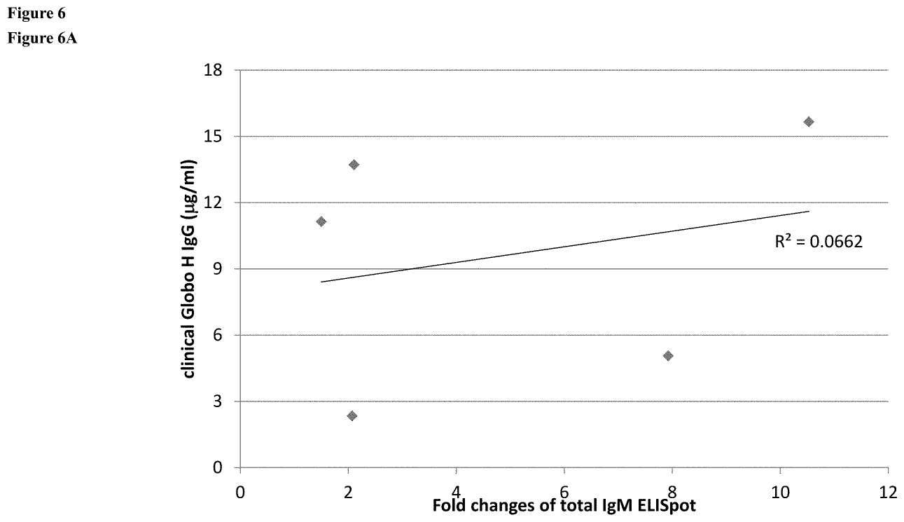

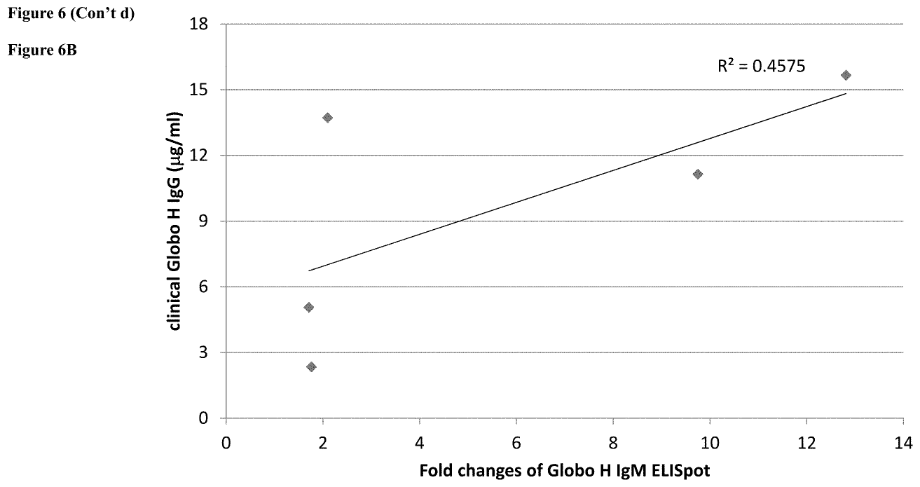

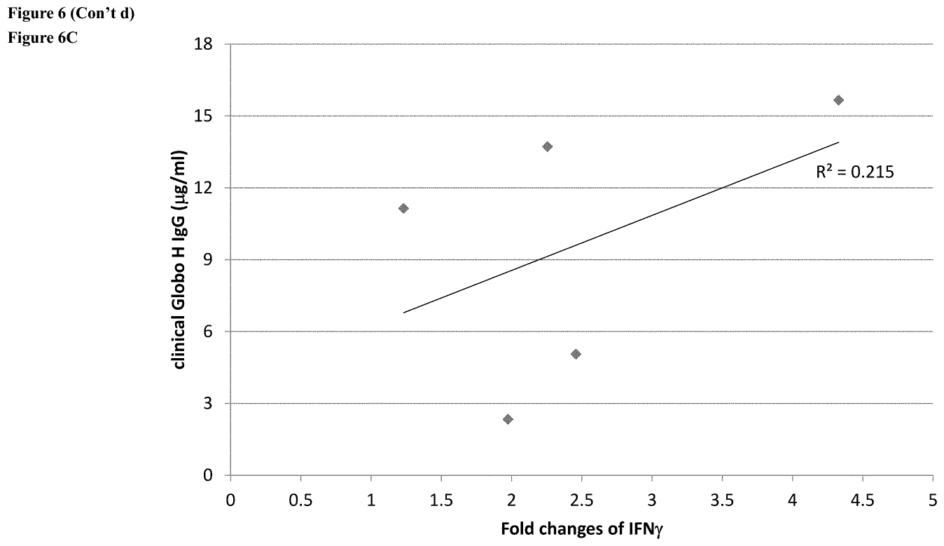

In addition, correlation of patients' maximum serum anti-Globo H IgG concentration (μg/mL) in comparison with either total IgM, anti-Globo H IgM, and IFN-γ cytokine induction are presented in FIGS. 6A, 6B, and 6C respectively.

Overall, results showed more obvious correlation with anti-Globo H IgM than anti-Globo H IgG. Patients may have delayed IgG response where correlation cannot be observed yet.
Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.
All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

1. A method of obtaining information that can be used to determine and predict the humoral immune response of patient suspected of having cancer for a carbohydrate antigen, comprising the steps of:
(a) obtaining a sample from the patient,
(b) cultivating a cell from the sample,
(c) exposing the cell ex vivo to one or more antigens,
(d) identifying the patient is a responder or a non-responder; and
(e) continuing a further treatment if the patient is a responder.

2. The method of claim 1, wherein the carbohydrate antigen is a Globo series antigen.

3. The method of claim 2, wherein the Globo series antigen is stage-specific embryonic antigen-4 (SSEA-4), stage-specific embryonic antigen-3 (SSEA-3) or Globo H.

4. The method of claim 1, wherein the cancer is a Globo series antigen expressing cancer.

5. The method of claim 4, wherein the Globo series antigen expressing cancer is sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testical cancer, buccal cancer, oropharyngeal cancer, laryngeal cancer or prostate cancer.

6. The method of claim 1, wherein the sample consists of blood, lymph node fluid, tumor biopsy or tissue culture.

7. The method of claim 1, wherein the cell is a human peripheral blood mononuclear cell (PBMC), a normal cell, a cancer cell, a stem cell or a cancer stem cell.

8. The method of claim 1, wherein the humoral immune response is an IgM, IgG, or a cytokine.

9. The method of claim 8, wherein the cytokine is human growth hormone, parathyroid hormone, glycoprotein hormone, fibroblast growth factor, TNF-α, TNF-β, vascular endothelial growth factor, integrin, thrombopoietin (TPO), nerve growth factor, platelet-growth factor, TGF-α, TGF-β, interferon-α, interferon-β, interferon-γ, macrophage-CSF (M-CSF), granulocyte-macrophage-CSF (GM-CSF), granulocyte-CSF (G-CSF), IL-1, IL-la, IL-2, IL-3, IL-4, IL-5, IL-6, IL-7, IL-8, IL-9, IL-10, IL-11 or IL-12.

10. A method for evaluating responsivity of a subject to a carbohydrate antigen or its immunogenic fragment, comprising:
(a) obtaining a peripheral blood mononuclear cell from the subject;
(b) exposing the cell ex-vivo with the carbohydrate antigen or its immunogenic fragment in an Enzyme-Linked ImmunoSpot assay performed by using carbohydrate antigen-coated plate; and
(c) determining a biological activity according to the assay to evaluate the responsivity.

11. The method of claim 10, wherein the carbohydrate antigen or its immunogenic fragment is a Globo series antigen.

12. The method of claim 11, wherein the Globo series antigen is stage-specific embryonic antigen-4 (SSEA-4), stage-specific embryonic antigen-3 (SSEA-3) or Globo H.

13. The method of claim 10, wherein the carbohydrate antigen or its immunogenic fragment is conjugated with a carrier protein.

14. The method of claim 13, wherein the carrier protein comprises KLH (Keyhole limpet hemocyanin) or DT-CRM 197 (diphtheria toxin cross-reacting material 197).

15. The method of claim 10, wherein the carbohydrate antigen is formulated with an adjuvant into a cancer vaccine.

16. The method of claim 15, wherein the adjuvant comprises QS-21, saponin, Freund's adjuvant, α-galactosyl-ceramide (α-GalCer) adjuvant, OBI-821, OBI-834, or a combination thereof.

17. The method of claim 15, wherein the cancer is a Globo series antigen expressing cancer.

18. The method of claim 17, wherein the Globo series antigen expressing cancer is sarcoma, skin cancer, leukemia, lymphoma, brain cancer, glioblastoma, lung cancer, breast cancer, oral cancer, head-and-neck cancer, nasopharyngeal cancer, esophagus cancer, stomach cancer, liver cancer, bile duct cancer, gallbladder cancer, bladder cancer, pancreatic cancer, intestinal cancer, colorectal cancer, kidney cancer, cervix cancer, endometrial cancer, ovarian cancer, testical cancer, buccal cancer, oropharyngeal cancer, laryngeal cancer or prostate cancer.

19. The method of claim 15, wherein the cancer vaccine is OBI-822/821, OBI-833/821, OBI-833/834, OBI-866/821 or OBI-866/834.

20. The method of claim 10, wherein the biological activity comprises production of IgM, IgM against the carbohydrate antigen, cytokine, or a combination thereof.

2321. The method of claim 20, wherein the biological activity is determined by spot number of the assay.

2422. The method of claim 10, wherein the responsivity is evaluated by comparing the biological activity with a threshold value and the subject is identified as responsive to the carbohydrate antigen if the biological activity exceeds the threshold value.

23. A method for determining the therapeutic efficacy of a cancer vaccine in a patient, comprising:
(a) providing a sample from the patient;
(b) cultivating a cell collected from the sample;
(c) contacting the cell and assaying the binding of antibodies bound to the antigens; and
(d) determining the therapeutic effect of the antineoplastic agent.

24. The method of claim 23, wherein the sample consists of blood, lymph node fluid, tumor biopsy or tissue culture.

25. The method of claim 23, wherein the antineoplastic agent comprising a vaccine composed of a carbohydrate antigen or its immunogenic fragment conjugated with a carrier protein.

26. The method of claim 25, wherein the carbohydrate antigen or its immunogenic fragment comprising Globo H, Stage-specific embryonic antigen 3 (SSEA-3) or Stage-specific embryonic antigen 4 (SSEA-4).

27. The method of claim 25, wherein the carrier protein comprising KLH (Keyhole limpet hemocyanin) or DT-CRM 197 (diphtheria toxin cross-reacting material 197)

28. The method of claim 23, wherein the treatment further comprising an additional adjuvant.

29. The method of claim 28, wherein the adjuvant is selected from QS-21, saponin, Freund's adjuvant, α-galactosyl-ceramide (α-GalCer) adjuvant, OBI-821, OBI-834, or a combination thereof.

30. The method of claim 25, wherein the vaccines are OBI-822/821, OBI-833/821, OBI-833/834, OBI-866/821 or OBI-866/834.
